(19) United States
(12) Patent Application Publication
(10) Pub. No.: US 2004/0210277 A1
(43) Pub. Date: Oct. 21, 2004

(54) Laser and light emitting diode body irradiator method and apparatus

(76) Inventors: Hans Becker, Dubuque, IA (US); Robert E. Moroney, Aurora, CO (US)

Correspondence Address: SHERIDAN ROSS PC, 1560 BROADWAY, SUITE 1200, DENVER, CO, 80202

(21) Appl. No.: 10/806,877

(22) Filed: Mar. 22, 2004

Related U.S. Application Data
(60) Provisional application No. 60/463,637, filed on Apr. 16, 2003.

(51) Int. Cl. A61N 1/00

(52) U.S. Cl. 607/88

(57) ABSTRACT

The present invention is directed to irradiating tissue using LED and laser light sources. A number of different head units having different light sources may be interconnected to a power supply unit. Memory is provided for storing user defined output programs. According to the identity of the head unit interconnected to the power supply, a different output program stored in the power supply may be selected and provided to the head unit. In connection with head units having multiple light sources, the power supply may provide power to operate different light sources at different frequencies simultaneously. The power supply may incorporate rechargeable batteries. The head units may include spot laser light sources providing substantially collimated light, and/or LED sources providing diffuse light.

### CROSS REFERENCE TO RELATED APPLICATION

This application claims the benefit of U.S. Provisional Application No. 60/463,637, filed Apr. 16, 2003, the entire disclosure of which is hereby incorporated herein by reference.

### FIELD OF THE INVENTION

The present invention relates to irradiation of animal tissue with light in connection with therapeutic purposes. In particular, the present invention relates to providing light of a selected wavelength or wavelengths to animal tissue at selected frequencies.

### BACKGROUND OF THE INVENTION

The cells of living bodies are affected by light. For instance, the stimulation of tissue using low energy laser and light emitting diode (LED) radiation has been investigated in connection with the promotion of the healing of wounds in animal, including human, tissue. For example, studies have investigated the use of powerful light emitting diodes in connection with wounds that are often particularly hard to heal, including diabetic skin ulcers, serious burns, and severe oral sores caused by chemotherapy and radiation. In addition to the use of light in connection with healing wounds, light stimulation of human bodies has been studied in connection with nerve regeneration, biomodulation, cellular oxygenation, and cellular detoxification. It is believed that such light therapy is or may be effective in connection with such uses because it stimulates cell growth and cell activity.

Light sources that have been considered for use in connection with light therapy include light emitting diode (LED), low level laser therapy (LLLT), or low power laser therapy (LPLT). In connection with irradiating tissue using light from an LED or from a laser, it is important to prevent accidental exposure, as such exposure can be dangerous, especially to the eyes. However, devices that provide appropriate safeguards against accidental exposure have not been available. In addition, conventional devices have been limited in the amount of control over the output of the light, and the available wavelengths of light. Furthermore, devices heretofore available have not provided for the application or the convenient selection of different application parameters. For example, conventional light therapy devices have not allowed users to conveniently select light of different output wavelengths, or to control the frequency at which the light is pulsed. As a further example, conventional light therapy devices have not provided for the convenient use of different frequencies of operation and overall operating times in connection with light sources having different output wavelengths.

### SUMMARY OF THE INVENTION

The present invention is directed to solving these and other problems and disadvantages in the prior art.

According to embodiments of the present invention, different hand-held head units, each having a different selection of light sources, is provided. Each head unit may be selectively interconnected to a power supply. Head units may differ from one another in the number and/or type of included light sources. For example, head units may include different selections or combinations of light emitting diode or laser light sources. In addition, the wavelengths of light provided by the sources, and therefore different selections or combinations of output wavelengths, may be provided by different head units.

In accordance with further embodiments of the present invention, the power supply may be operated to provide to the light source or sources of the head unit a signal that is pulsed on and off at a selected frequency. In addition, the power supply may be operated to provide a different signal to each light source or group of light sources included as part of an interconnected head unit, allowing different light sources in the head unit to be pulsed on and off at different frequencies. In accordance with other embodiments of the present invention, the light source or sources included as part of a head unit interconnected to the power supply may each be operated by providing a pulsed signal for predetermined periods of time. The frequency of a signal provided to a light source or group of light sources included as part of a head unit may also be varied over time. Still other embodiments of the present invention allow light sources in a head unit to be operated continuously.

Embodiments of the present invention allow a therapist to store a number of different output programs that can be selected for use with different heads. Output parameters that may be stored as part of output programs include different output frequencies for different light sources and operating times. In accordance with other embodiments of the present invention, the power supply may receive an identification signal from an interconnected head unit, and may select a power supply program comprising a predetermined pulse frequency or frequencies and operating times accordingly. Therefore, a therapist need not manually program the power supply in order to provide a power signal to the light sources of a head unit at an appropriate frequency or frequencies and for an appropriate time period. In accordance with additional embodiments of the present invention, programming options available to a user are determined by the capabilities of a head unit interconnected to the power supply.

In accordance with still other embodiments of the present invention, a head unit that includes a laser or other high intensity device as a light source may be provided with an interlock or key switch. Accordingly, the inadvertent or unauthorized use of such a head unit can be prevented or deterred, reducing the risk of injury from the high intensity light.

Additional features and advantages of the present invention will become more apparent from the detailed description, particularly when taken together with the accompanying drawings.

### BRIEF DESCRIPTION OF THE DRAWINGS

FIG. 1 illustrates a laser and light emitting diode irradiator system in accordance with embodiments of the present invention;

FIG. 2 is a cross section of a head unit comprising light emitting diode light sources in accordance with embodiments of the present invention;

FIG. 3A is an output end view of the head unit of FIG. 2;

FIG. 3B is a power cord receptacle end view of the head unit of FIG. 2;

FIG. 4 is a cross section of a head unit comprising laser light sources in accordance with embodiments of the present invention;

FIG. 5A is an output end view of the head unit of FIG. 4;

FIG. 5B is a power cord receptacle end view of the head unit of FIG. 4;

FIG. 6 is a circuit diagram of the head unit of FIG. 2;

FIG. 7 is a circuit diagram of the head unit of FIG. 4;

FIG. 8 is a circuit diagram of a power supply unit in accordance with embodiments of the present invention;

FIG. 9 is a flow diagram depicting aspects of the operation of a laser and light emitting diode irradiating system in accordance with embodiments of the present invention;

FIGS. 10A and 10B illustrate example power supply displays in accordance with embodiments of the present invention; and

FIG. 11 is a flow diagram depicting additional aspects of a laser and light emitting diode irradiating system in accordance with embodiments of the present invention.

### DETAILED DESCRIPTION

In accordance with embodiments of the present invention, methods and apparatuses for providing a therapeutic laser and light emitting diode irradiating system are disclosed.

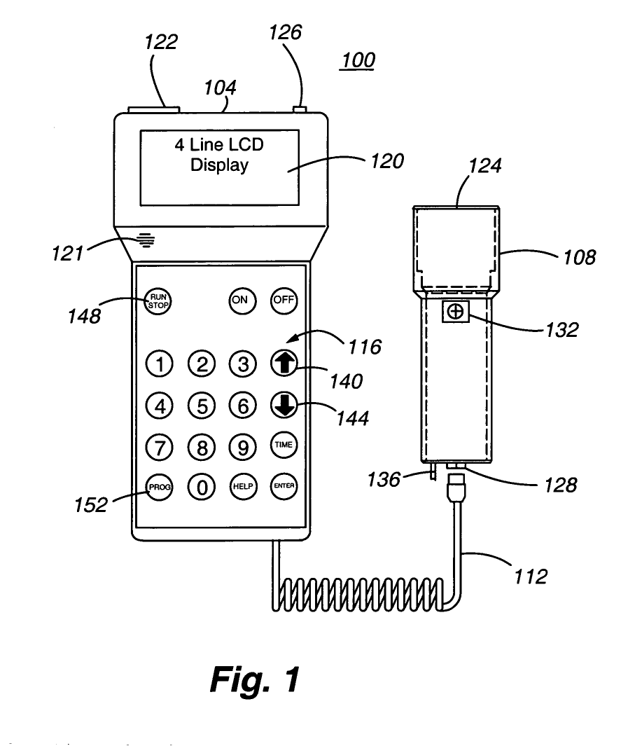

In FIG. 1, a laser and light emitting diode irradiating system 100 in accordance with an embodiment of the present invention is depicted. In general, the system includes a power supply unit 104 and a head unit 108. A power supply cord 112 is provided for interconnecting the power supply 104 to the head unit 108.

The power supply 104 may be provided with a control panel 116 for receiving input from an operator, such as a therapist. In addition, the power supply 104 may include an output device, such as a multiple line visual display 120. The power supply 104 may additionally be provided with an audible output device 121, and/or with an input/output port 122 suitable for interconnecting the power supply 104 to a general purpose computer or to a communication channel.

The power supply 104 may be battery operated. In accordance with an embodiment of the present invention, the battery is rechargeable. A plug 126 may be provided to connect the power supply to an AC power source for recharging the battery, or for powering the power supply 104 in place of or in addition to the battery.

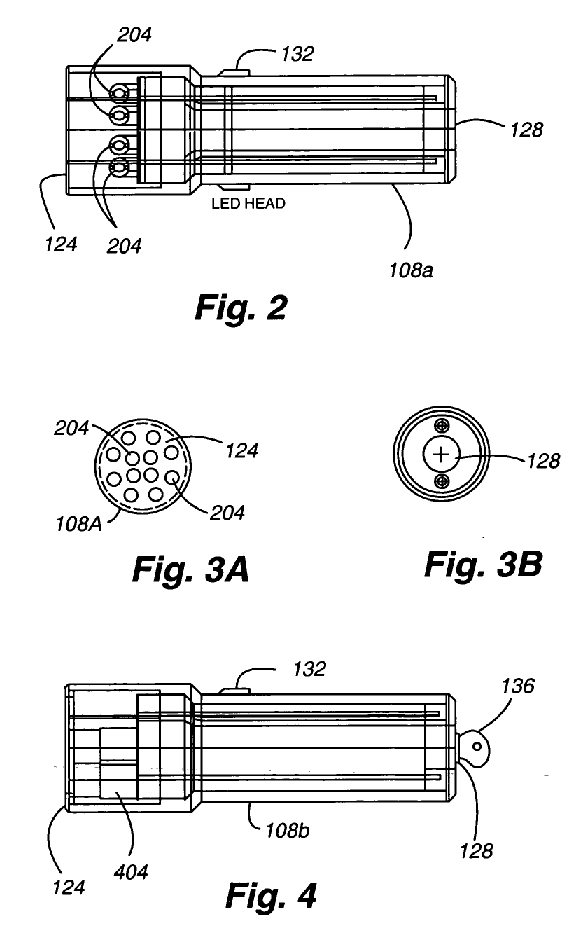

The control panel 116 may be operated to select output parameters of the power supply 104. For example, the frequency of the signal provided to a light source included as part of the head unit 108 may be selected. That is, the frequency at which a power signal supplied to the head unit or individual or groups of light sources (e.g., light sources 204 and 404 shown in FIGS. 2 and 4) is switched on and off (or pulsed) may be selected. In accordance with embodiments of the present invention, the output frequency provided to a light source or a group of light sources may be selected from a range of frequencies. For example, a frequency from 0.1 Hz to 5999.0 Hz may be selected. In accordance with embodiments of the present invention, the duty cycle of the pulsed output signal may be 50%. In addition, embodiments of the present invention may allow light sources in a head unit to be operated continuously. The control panel may also be operated to allow the operator to select a total amount of time that output is provided to the head unit 108. As will be described in greater detail elsewhere herein, a selected output frequency or frequencies and total output time may comprise an output program. Furthermore, a number of user-defined output programs may be stored in a power supply unit 104 for use with different head units 108 and/or for different therapeutic purposes. In addition, pre-provisioned programs, for supplying output signals to the light sources of the head unit 104 having different frequencies (or combinations of frequencies) and times may be selected.

The head unit 108 includes light sources 204, 404 (see FIGS. 2 and 4) at a first end 124. An interconnect or receptacle 128 may be provided to selectively interconnect the head unit 108 to the power supply cord 112 and in turn to the power supply 104. Accordingly, different head units 108 may be conveniently interconnected to the power supply 104. The head unit 108 may additionally include a power switch 132, to allow the operator to position the head unit 108 as desired and commence operation of the light sources, without needing to access the control panel 116 of the power supply 104. Embodiments of the present invention may additionally include an interlock or key switch 136, to prevent the inadvertent or unauthorized use of the head unit 108. The key switch 136 may require a mechanical or electronic key, such as the entry of a password or code either manually or through a read device, such as a magnetic card reader, before the head unit 108 can be operated. The provision of a key switch 136 is believed to be particularly desirable when the light source of the head unit 108 comprises a potentially dangerous laser.

As is described in greater detail elsewhere in the present disclosure, the head unit 108 may provide an identification signal to the power supply 104. The identification signal allows the power supply to provide an appropriate output signal to the head unit. For example, outputs provided by the power supply 104 that are not connected to any light source of an interconnected head unit 108 are not energized. As a further example, the polarity of the output from the power supply 104 is selected to correspond to the requirements of an interconnected head unit 108. As still another example, the output is provided in accordance with parameters determined by an appropriate program. In accordance with an embodiment of the present invention, the identification signal is provided automatically to the power supply 104. For example, the identification signal may be provided at the time the head unit 108 is interconnected to the power supply 104 by the power cord. As an additional example, the identification signal may be sent to the power supply 104 after the power supply 104 has provided an interrogation signal or has supplied power to the head unit 108.

With reference now to FIGS. 2, 3A and 3B, a head unit 108a in accordance with an embodiment of the present invention is depicted. As seen in FIGS. 2, 3A and 3B, the head unit 108a may comprise a number of light emitting diodes (LEDs) or LED light sources 204 at a first end 124 of the head unit 108a. The LEDs 204 may produce light of a single wavelength or a range of wavelengths projected over a relatively large area. The wavelength or wavelengths produced by an LED 204 may range from the ultraviolet to the infrared. An interconnect 128 is provided for connecting the head unit 108a to a power supply cord 112 and in turn to a power supply 104. A power switch 132 may be provided to control the operation of the head unit 108a.

The LEDs 204 may be selected so that each one of the LEDs 204 generates light of the same wavelength or range of wavelengths. Alternatively, different wavelengths may be generated. For example, a head unit 108 such as the head unit 108a illustrated in FIGS. 2, 3A and 3B having twelve LEDs may include a first group of 3 LEDs 204 that produce light having a first wavelength, a second group of 3 LEDs 204 that produce light having a second wavelength, a third group of 3 LEDs 204 that produce light having a third wavelength, and a fourth group of 3 LEDs 204 that produce light having a fourth wavelength. Alternatively, each of the LEDS 204 may output light at the same wavelength or range of wavelengths. In addition, the twelve LEDs 204 of the head unit 108 may be separately operated in four groups of three LEDs 204. Exemplary output wavelengths for light sources comprising LEDs 204 include 628 nm red LEDs and 850 nm clear LEDs. Furthermore, exemplary LEDs 204 may each have a power output of less than 5 mW, such as an output of 4.5 mW, and may form non-convergent beams. As will be described in greater detail elsewhere in this disclosure, each group of LEDs 204 may be provided with a separate signal or power channel from the power supply, and thus may be operated at different times and/or at different frequencies.

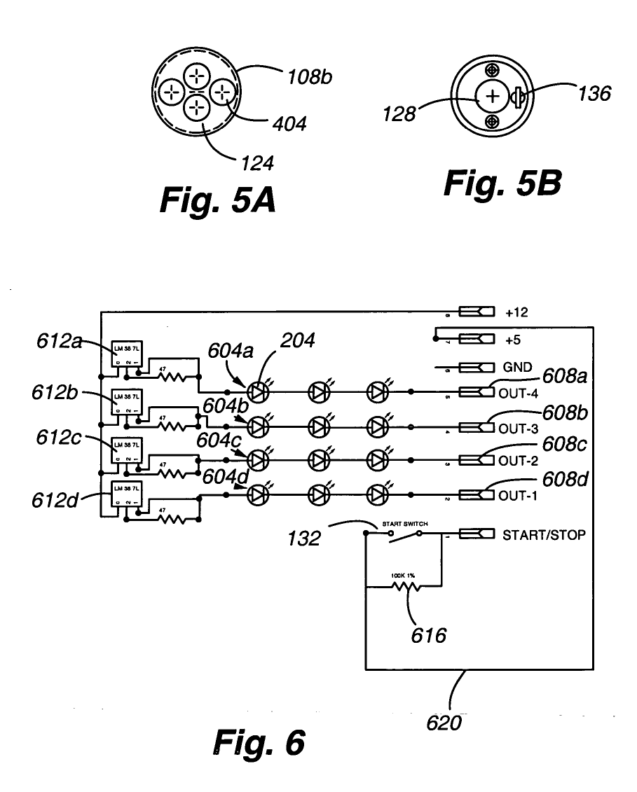

With reference now to FIGS. 4, 5A and 5B, a head unit 108b in accordance with another embodiment of the present invention is illustrated. As seen in FIGS. 4 and 5, the head unit may comprise a number of laser light sources 404 at a first end 124 of the head unit 108b. The sources of laser light 404 may comprise laser diodes or some other relatively low output device for producing substantially collimated light of a single wavelength or a limited number of wavelengths. For example, laser light sources 404 may provide light having a wavelength of 635 nm or 830 nm. The laser light sources 404 may be operated separately or as groups of more than one laser light source. Accordingly, output signals having different frequencies may be provided to some or all of the laser light sources 404. The laser light sources 404 may have a power output of 5 mW or less. For example, the power output may be 4.5 mW. An interconnect 128 is provided for connecting the head unit 108b to a power supply cord 112 and in turn to a power supply 104.

Accordingly, it should be appreciated that a first head unit 108a or a second head unit 108b can be selectively interconnected to a power supply 104. In addition to a power switch 132, the head unit 108b may provide an interlock or key switch 136, and an appropriate key must be provided to the key switch 136 before operation of the head unit 108b can commence. The key required in order to operate the head unit 108b may comprise a mechanical or electronic key, for example in the form of an access code. In embodiments requiring an access code, such code may be provided to a reader on or interconnected to the head unit 108b or power supply 104 in the form of a key card carried by the operator, it may be entered by the operator using the keypad 116 of an interconnected power supply 104, or it may be entered using an input device or devices provided as part of the head unit 108b.

FIG. 6 is a circuit diagram of the head unit 108a depicted in FIGS. 2, 3A and 3B. As shown in FIG. 6, the LED light sources 204 are divided into four groups 604a-d, each including three LEDs 204. Exemplary wavelengths of the light output by all or some of the LED light sources include 628 nm and 830 nm. A separate signal line 608a-d is associated with each of the groups 604. Accordingly, each group 604 of LED light sources 204 can be separately controlled by the power supply 104. Current regulators 612a-d may be provided for ensuring that a proper current is provided to each group 604 of light sources 204. Although FIG. 6 shows 4 groups 604 each having three LED light sources 204, it should be appreciated that different numbers and combinations of LED light sources 204 may be included in a head unit 108.

A resistor 616 having a selected resistance (for example 100K ohms) is provided across the power switch 132. By assigning a different resistor value to each compatible head unit 108, the identity of a particular head unit interconnected to the power supply 104 can be determined by the voltage drop within the start/stop circuit 620.

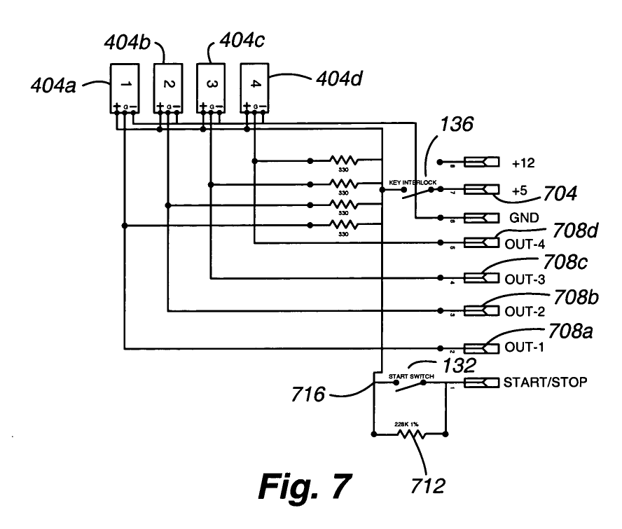

FIG. 7 is a circuit diagram of the head unit 108b depicted in FIGS. 4, 5A and 5B. As shown in FIG. 7, the laser light sources 404 and are interconnected to a power supply contact 704 through the interlock 136. Each laser light source 404 has a pair of power supply inputs (+ and −) and a signal input (S). Each signal input is interconnected to a separate signal line 708a-d, allowing each laser light source 404 to be separately controlled by the power supply 104. Exemplary wavelengths of the light output by all or some of the laser light sources 404 include 635 nm and 830 nm.

A resistor 712 having a selected resistance (for example 220K ohms) is provided across the power switch 132. As noted above, by assigning a different resistor value to different head units 108, the identity of a particular head unit interconnected to the power supply 104 can be determined by the voltage drop within the start/stop circuit 716.

The circuit for the head unit 108b having laser light sources 404 additionally includes an interlock switch 136. As can be seen from FIG. 7, the laser light sources 404 cannot be operated if the interlock switch 136 is open. In addition, it will be noted by comparing the head unit 108 circuits in FIGS. 6 and 7 that the signal lines 608 for the LED head unit 108a in FIG. 6 have a negative polarity, while the signal lines 708 for the laser head unit 108b in FIG. 7 have a positive polarity. Accordingly, in such an embodiment, an LED head unit 108a cannot be operated by an output from the power supply 104 intended for a laser head unit 108b, and vice versa.

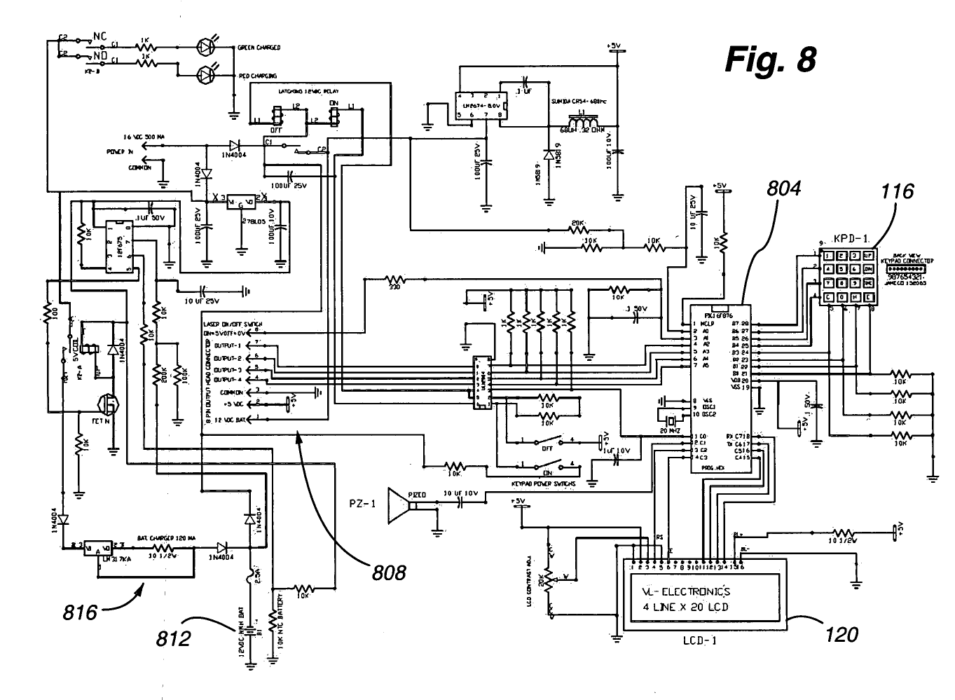

With reference now to FIG. 8, a circuit diagram of a power supply 104 in accordance with an embodiment of the present invention is illustrated. As shown in FIG. 8, a controller 804 is provided for controlling the output signal or signals provided at the outputs 808 of the controller 804. When a head unit 108 is interconnected to the power supply cord 112, one or more of the outputs 808 is interconnected to a corresponding signal line 608 or 708. The power supply 104 also includes a rechargeable battery 812 and an associated charger 816.

In accordance with an embodiment of the present invention, the controller or microcontroller 804 comprises a PIC16F876 microprocessor. The controller 804 may be programmed with a number of different programs such that an output signal at each output 808 provides a signal at a selected frequency and for a selected period of time. For example, embodiments of the present invention may provide 25 memory locations capable of storing four different output frequencies for controlling four separate light sources or groups of light sources in a head unit 108, and a program operating time. A particular output program may be selected in response to an input entered by an operator using the numeric keypad 116, or in response to a voltage drop detected in connection with the start control circuit of an interconnected head unit 108. For example, in response to an identification signal received from an interconnected head unit 108, a program corresponding to the interconnected head unit 108 may be selected. An example of an identification signal in accordance with an embodiment of the present invention is a voltage drop across a start/stop circuit introduced by a resistor 616 or 712.

The battery 812 may comprise a battery rated at 12.2 Volts and 1.6 Amperes for a power output of 19.5 Watts. Furthermore, the battery 812, in an exemplary embodiment, is rated for four hours of continuous operation. In accordance with embodiment of the present invention, the total power output of the power supply 104 may be about 5 Watts.

As an illustrative example, if a head unit 108a as illustrated in FIGS. 2, 3A. 3B and 6 is interconnected to the power supply, a program may be selected that provides a first output signal via a first output 808 at a first frequency to the first group 604a of LEDs 204, a second output signal via a second output 808 at a second frequency to the second group 604b of LEDs 204, a third output signal via a third output 808 at a third frequency to the third group 604c of LEDs 204, and a fourth output signal via a fourth output at a fourth frequency to the fourth group 604d of LEDs 204. Furthermore, the first, second, third and fourth output signals may all be provided simultaneously for a programmed period of time.

As another example, if a head unit 108b as illustrated in FIGS. 4, 5A, 5b and 7 is interconnected to the power supply 104, a program may be selected that supplies a first output signal via a first output 808 at a first frequency to the first laser light source 404a for a first period of time, at a second frequency to the second laser light source 404b for the first period of time, a third output signal via a third output 808 at a third frequency to the third laser light source 404c for the first period of time, and a fourth output signal via a fourth output 808 at a fourth frequency to the fourth laser light source 404d for the first period of time. In either of the illustrations provided above, the first, second, third and fourth frequencies may be different or the same. An example range of frequencies that may include each output signal frequency is from 0.1 to 30,000 Hz. In accordance with other embodiments of the present invention, the range of frequencies that can be supplied ranges from 0.1 to 5999.0 Hz. An example range of times that each output signal is provided is from 1 second to 5 minutes. The light sources 204 and 404 may further be operated simultaneously and/or sequentially. As can be appreciated by one of skill in the art, the selected program may be started each time the start button 132 on the head unit 108 is activated.

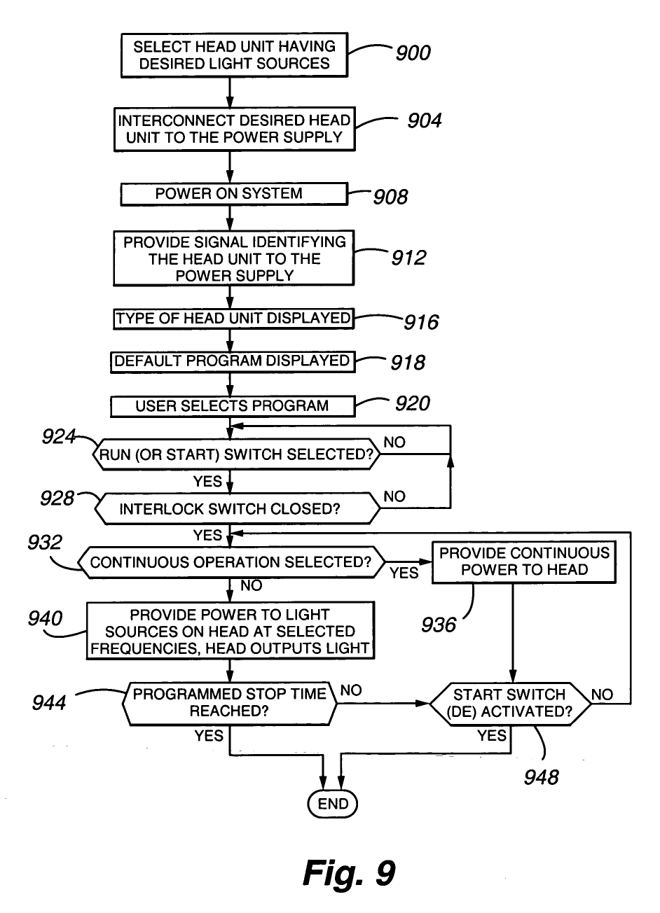

With reference now to FIG. 9, a method for irradiating animal tissue in accordance with an embodiment of the present invention is illustrated. Initially, at step 900, a head unit 108 having the desired light sources 204 or 404 is selected. The selected head unit 108 is then interconnected to a power supply 104 (step 904). At step 908, the system is powered on, and at step 912 the identity of the head unit 108 is communicated to the power supply 104. For example, an identity signal may be provided by a resistor having a predetermined value that is included in a start/stop circuit of the head unit 108, by a coded identification signal provided by the head unit 108 in response to an interrogation signal from the power supply, or by an operator who manually enters information identifying the head unit 108. Alternatively, an identity signal may be provided to the power supply by the head unit 108 when the head unit is interconnected to the power supply 104 by a power supply cord 112.

At step 916, the type of head unit 108 interconnected to the power supply 104 is displayed in the display 120. The information shown by the display 120 of the head unit 104 may include an identification of the particular model of head unit 108 that has been interconnected to the power supply 104. For instance, a head unit 108a illustrated in FIGS. 2, 3A, 3B and 6 may be identified as “HEAD TYPE=LED”. As a further example, the head unit 108b illustrated in FIGS. 4, 5A, 5B and 7 may be identified as “HEAD TYPE=LASER−4H.” As yet another example, a head unit 108 having two different laser sources may be identified as “HEAD TYPE=LASER−2H.” In general, the type of head unit 108 is displayed for a short period of time, for example three seconds.

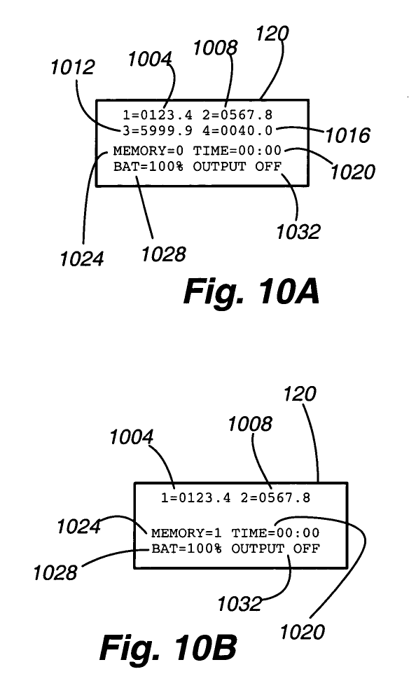

After displaying an identification of the head unit 108, a default program screen for the interconnected head unit is displayed (step 918). In general, the default program screen displayed will include a default output frequency for each light source or group of light sources included in that type of head unit 108. Accordingly, for head units 108 such as those illustrated in FIGS. 2-7, four separate frequencies, shown in separate frequency fields 1004, 1008, 1012 and 1016 (see FIG. 10A) will be displayed. As shown in the time field 1020 in FIGS. 10A and 10B, the default program may have a default run time of zero. The display 120 may also show the memory location 1024 of the default program or of a selected program, the battery status 1028, and the output status 1032. As a further example, a head unit 108 having only two light sources or groups of light sources will include only first 1004 and second 1008 frequencies, and a default time of zero (see FIG. 10B). The default program may be different for different types of head units. For example, different frequencies may be used for LED head units (e.g., 108a) than for laser head units (108b). Also, if the identifying signal identifies a particular head unit (i.e., rather than identifying the head unit 108 more generally by number of light sources or light source groups and/or type of light sources), the default program may include frequencies that are particular to that head unit 108.

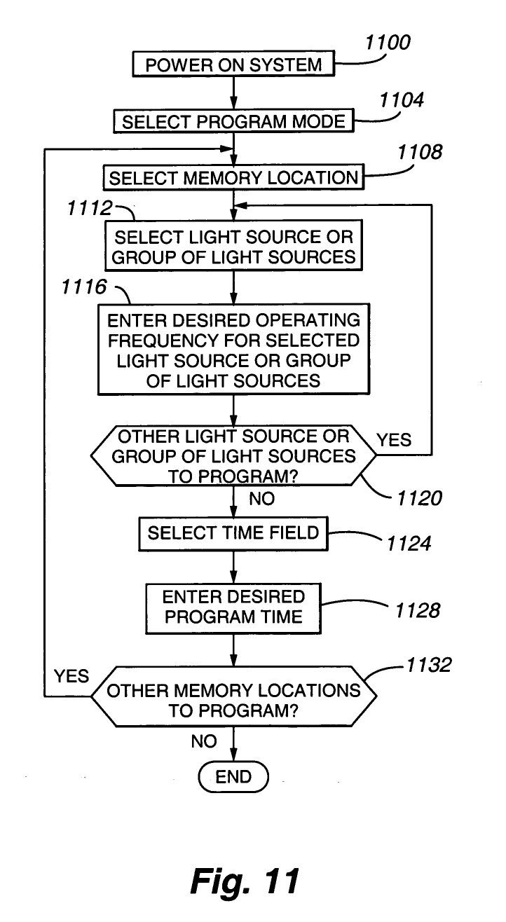

At step 920, the user may select a program that has already been entered into the power supply 104, or the user may enter a new program (see FIG. 11 and the accompanying discussion). In order to select a program that has already been entered, the user may select the desired program by pressing the advance key 140 or the reverse key 144 until the desired program or program number is displayed, and then pressing the enter key.

At step 924, a determination is made as to whether a start signal from the head unit 108 has been received by the power supply 104. A start signal may be entered by a user by pressing the “run stop” button 148 on the power supply 104 or the power switch 132 on the head unit 108. If no start signal is received, the procedure may idle at step 924. Once a start signal is received the process proceeds to step 928 where a determination is made as to whether any interlock switch 136 associated with the head unit 108 has been closed. If the head unit has an interlock switch 136 and that switch 136 has not been closed, the method returns to step 924 (i.e. both start switch and interlock switch 136 must be closed in order for an interlock switch 136 equipped head unit 108 to operate). If the interlock switch 136 is closed, or if no such switch is provided as part of the head unit 108, a determination is made at step 932 as to whether continuous operation of the light sources included in the head unit 108 has been selected. In accordance with embodiments of the present invention, continuous operation (i.e., supplying an output signal that is not pulsed such that the light sources are operated at a 100% duty cycle) is selected by holding down the run stop button 148 or the power switch 132 for more than one second. If it is determined that continuous operation has been selected, continuous power is provided to the head (step 936).

If continuous operation has not been selected, for example the user has pressed the run stop button 148 or the power switch 132 for less than one second, power is provided to the light sources on the head unit 108 at the selected frequencies, and the head unit 108 outputs light (step 940), pulsed at the selected frequencies. After the provision of power to the light sources has begun at step 940, a determination is made as to whether the programmed stop time has been reached (step 944). If the programmed stop time has been reached, the process ends.

If at step 944 it is determined that the programmed stop time has not been reached, a determination is made as to whether the start switch has been activated (step 948). For instance, according to embodiments of the present invention, the output of light from a head unit 108 can be discontinued by pressing the run/stop button 148 or the start/stop button 132 a second time for less than one second. Similarly, after the provision of continuous power to the head unit 108 has commenced at step 936, a determination is made at step 948 as to whether the start switch has been deactivated. In particular, embodiments of the present invention allow a continuous output from the head unit 108 to be maintained until the user releases the run/stop button 148 or the start/stop button 132. If it is determined that programmed operation or continuous operation has not been stopped, the process returns to step 932. If the programmed or continuous operation has been discontinued or deactivated, the process ends.

With reference now to FIG. 11, additional aspects of the operation of embodiments of the present invention are depicted. Initially, at step 1100, the system 100 is powered on. At step 1104, the program mode is selected. In accordance with embodiments of the present invention, the program mode is selected by pressing the program button 152 on the power supply 104 (See FIG. 1).

At step 1108, a memory location for programming is selected. A memory location may be selected by depressing the advance key 140 and/or the reverse key 144 until the desired memory location number is displayed by the power supply 104. A light source or group of light sources is then selected (step 1112). In accordance with embodiments of the present invention, the number of light sources or groups of light sources that can be separately controlled is determined by the identity of the head unit 108 interconnected to the power supply 104 when the power supply 104 is being programmed. Alternatively, the maximum number of separate light sources or groups of light sources that can be controlled by the power supply 104 is displayed, in which case the user would not enter values for light sources or groups of light sources that are not provided by a head unit 108 for which the user intends the program being entered. For example, if the user is entering a program at a memory location for a head unit 108 that only supports two separately controllable light sources, the user would enter output frequencies for only the first and second memory input fields 1004 and 1008. At step 1116, a desired operating frequency for each light source or group of light sources is entered.

At step 1120, a determination is made as to whether other light sources or groups of light sources remain to be programmed. If the program being entered is intended for a head unit having additional separately controllable light sources or groups of light sources, the process returns to step 1112. If no other light sources or groups of light sources are to have an assigned output frequency, the user selects the time field 1020 (step 1124). At step 1128, the user enters the desired program output time or run time in the time field 1020.

At step 1132, a determination is made as to whether other memory locations remain to be programmed. If the user desires to enter programs at other memory locations, the process returns to step 1108. If the user does not desire to program any more memory locations, or to reprogram any memory locations, the process ends.

Although the disclosure provided herein has described particular embodiments, it should be appreciated that the present invention is not so limited. For example, head units 108 that include both LED 204 and laser 404 light sources may be provided. Also, head units having any number of light sources 204 and/or 404 may be provided. Furthermore, although embodiments that include four separate output channels have been described, a lesser or greater number of channels may be provided.

The foregoing discussion of the invention has been presented for purposes of illustration and description. Further, the description is not intended to limit the invention to the form disclosed herein. Consequently, variations and modifications commensurate with the above teachings, within the skill and knowledge of the relevant art, are within the scope of the present invention. The embodiments described hereinabove are further intended to explain the best mode presently known of practicing the invention and to enable others skilled in the art to utilize the invention in such or in other embodiments and with various modifications required by their particular application or use of the invention. It is intended that the appended claims be construed to include the alternative embodiments to the extent permitted by the prior art.

What is claimed is:

1. A method of irradiating a body, comprising:
providing a first head unit having at least a first light source, said at least a first light source including at least a first one of a light emitting diode and a laser;
interconnecting said first head unit to a power supply; and
supplying an output signal having at least a first frequency to said a least a first light source of said first head unit.

2. The method of claim 1, further comprising providing identifying information regarding said first head unit to said power supply.

3. The method of claim 1, wherein said output signal having at least a first frequency is selected in response to a selection of a first program.

4. The method of claim 3, further comprising:
supplying an output signal to said at least a first light source of said first unit having at least a second frequency, wherein said output signal having at least a second frequency is selected in response to a selection of a second program.

5. The method of claim 2, wherein said step of providing identifying information comprises interposing a first resistance in a power supply circuit of said first head unit.

6. The method of claim 1, further comprising:
providing a second head unit comprising at least a second light source, said second light source including at least a second one of a light emitting diode and a laser;
disconnecting said first head unit from said power supply;
interconnecting said second head unit to said power supply; and
supplying an output signal having at least a second frequency to said at least a second light source of said second head unit.

7. The method of claim 1, wherein said first head unit includes a number of light sources, wherein an output signal having said first frequency is supplied to a first of said light sources, said method further comprising:
supplying an output signal having a second frequency to a second of said light sources.

8. The method of claim 7, wherein said output having a first frequency and said output having a second frequency are supplied to said respective light sources simultaneously.

9. The method of claim 7, wherein said output having a first frequency and said output having a second frequency are selected in response to selection of a first output program.

10. The method of claim 9, further comprising:
selecting a second output program; and
in response to said selecting a second output program, supplying output having a third frequency to said first of said light sources and output having a fourth frequency to said second of said light sources.

11. The method of claim 7, wherein a first of said light sources produces light having a first wavelength and a second of said light sources produces light having a second wavelength.

12. The method of claim 1, further comprising:
starting a timer when said output is initiated; and
after providing said output for a predetermined period of time, discontinuing said output.

13. The method of claim 1, wherein said first light source comprises a laser light source, said method further comprising, prior to said supplying an output signal, determining that a key interlock switch is closed.

14. A body irradiator device, comprising:
a first head unit, including:
at least one light source;
a power supply selectively interconnected to said first head unit, wherein said power supply provides an output signal to said at least one light source of said first head unit at:
a) at least a first frequency; and
b) for at least a first selected period of time.

15. The device of claim 14, wherein said first head unit further includes a number of light sources, wherein said power supply provides an output signal at said first frequency to a first one of said at least one light source, and wherein said power supply provides an output signal at a second frequency to a second one of said light sources.

16. The device of claim 14, wherein said at least one light source comprises at least one of a laser and a light emitting diode.

17. The device of claim 14, wherein said at least one light source comprises one of a Gallium Aluminum Arsenide laser diode and a light emitting diode.

18. The device of claim 14, wherein said light source has an output wavelength of one of 628 nm, 635 nm, 830 nm, and 850 nm.

19. The device of claim 14, wherein said first frequency within a range from 0.1 Hz to 5999.0 Hz.

20. The device of claim 14, wherein said power supply comprises a display operable to indicate a frequency at which power is being supplied to said at least one light source.

21. The device of claim 14, wherein said power supply is battery operated.

22. The device of claim 14, wherein said first head comprises a first circuit element for identifying said first head unit.

23. The device of claim 22, wherein said first circuit element comprises a first resistor located within a power supply circuit of said first head.

24. The device of claim 23, wherein said first head unit further comprises a power supply switch, wherein said first resistor is in parallel with said power supply switch.

25. The device of claim 14, further comprising:
a second head unit, including:
a light source comprising at least a first laser;
a power supply switch;
a key interlock switch; and
a resistor in parallel with said power supply switch.

26. A device for applying light to a body, comprising:
means for generating light;
means for receiving at least a first selected output frequency;
means for receiving at least a first selected output time;
means for outputting a power signal at said first selected output frequency and said first selected output time; and
means for interconnecting said means for outputting a power signal to said means for generating light, wherein said means for generating light is operated at said first selected output frequency for said first selected output time.

27. The device of claim 26, wherein said means for generating light comprises at least first and second light sources, wherein first and second output frequencies are received by said means for receiving at least a first output frequency, and wherein said first light source of said means for generating light is operated at said first selected frequency for said first selected output time and said second light source of said means for generating light is operated at said second selected frequency for said first selected output time.
